(19) United States
(12) Patent Application Publication
(10) Pub. No.: US 2002/0053099 A1
(43) Pub. Date: May 2, 2002

(54) New plants formed by microparticle bombardment with uncharacterised DNA

(76) Inventors: Robert Henry, Tuckombil (AU); Mahin Abedinia, Chapel Hill (AU); Anthony Bernar Blakeney, Lane Cove (AU); Laurence George Lewin, Leeton (AU)

Correspondence Address: MORGAN, LEWIS & BOCKIUS, L.L.P., 1701 Market Street, Philadelphia, PA, 19103-2921, US

(21) Appl. No.: 09/839,813

(22) Filed: Apr. 20, 2001

Related U.S. Application Data
(63) Continuation of application No. PCT/AU99/00897, filed on Oct. 18, 1999.

(30) Foreign Application Priority Data
Oct. 18, 1999 (AU) PCT/US00/24244
Oct. 22, 1998 (AU) PP6646

(51) Int. Cl. A01H 5/00; C12N 15/82

(52) U.S. Cl. 800/320.2; 435/468; 800/278; 435/419; 800/298; 800/320

(57) ABSTRACT

A method of plant gene transfer is provided which includes microprojectile bombardment using DNA directly isolated from a donor plant. Also provided are transgenic plants, cells and tissues thereof and progeny plants derived therefrom, the genomes of which have significant introgressions of the donor plant genome integrated therein.

### FIELD OF THE INVENTION

THIS INVENTION relates to a method of gene transfer applicable to plants. More particularly, the method relates to transfer of plant DNA by microprojectile bombardment. This invention also relates to transgenic plants, and in particular transgenic Oryza sativa (rice).

### BACKGROUND OF THE INVENTION

The transfer of desirable phenotypic traits between plants has traditionally been performed by selective breeding. Such breeding practices have been central to the development of efficient agricultural societies. More recently, recombinant DNA technology has revolutionized breeding practices, although the overall aims of conventional breeding and recombinant DNA technology are virtually identical. In fact, in hindsight it is now evident that traditional breeding practices provided a means whereby genetically-heritable phenotypic traits were introduced into plants, although in the absence of any knowledge of the genetic basis of heritability.

The underlying principle of modern recombinant DNA technology is that phenotype correlates with genotype. The resultant practical implication for genetic engineering is that transfer of a specific phenotypic trait can be achieved by transfer of a corresponding gene or genes. Accordingly, the one or more genes typically underlie phenotypic traits not normally exhibited by the plant.

As used herein, the plant which acts as a source of a gene is the “donor” and the plant into which the gene is introduced is the “recipient”. A donor and recipient may be genetically distinct by virtue of being members of different species, or different cultivars, breeds, races or strains, or by being different individual members of the same species, breed, race or strain. The plant resulting from gene transfer from said donor to said recipient is a “transgenic” plant.

It will also be appreciated that the transferred gene is not necessarily plant-derived. For example, transgenic plants have been made which express the bacterial Bt gene, the bacterial EPSP synthase gene and bacterial glufosinate (BASTA) detoxifying enzymes.

Generally, gene transfer methods applicable to genetic engineering have sought to achieve either transient or stable introduction of a gene. Stable expression occurs when the transferred gene is stably integrated into the genome of a recipient plant. It is stable integration of a transferred gene which allows genetic engineering to permanently and heritably modify plant genotype, and hence phenotype.

In plants, an obstacle to both transient and stable gene expression has been the lack of a generally applicable system for gene transfer. The earliest useful method, which in fact is still widely employed, is Agrobacterium mediated transformation. Agrobacterium tumifaciens is a naturally-occurring bacterial parasite of plants which harbours a tumour-inducing (Tᵢ) plasmid useful for introduction of genes into plants. Reference is made, for example, to International Publication No. WO 84/02920 in this regard. However, A. tumifaciens can be very selective in terms of infectable donor plants, and has proven to be virtually useless with monocotyledons such as cereals. This limited applicability has led to alternative means of transferring genes into plants.

Such alternative systems include electroporation, PEG-mediated transformation, microinjection and protoplast fusion. Although these systems have had some success, generally, they involve transfer of genes into cell suspensions or protoplasts, from which regeneration of plants has proven problematic (reviewed in Christou, Particle Bombardment Technology for Gene Transfer pp 71-99. Eds Ning-Sun Yang & Paul Christou, UWBC Biotechnical Resource Series, which is herein incorporated by reference)

A particularly important development has been in the area of “microprojectile bombardment”, alternatively known as “biolistic”, “particle” or “microparticle” bombardment. Whichever term is used, this approach generally involves coating microprojectiles with DNA, and accelerating the DNA-coated microprojectiles with a “particle gun” into suitable cells derived from a recipient plant. The microprojectiles are usually small, dense and made of biologically inert material such as gold. The bombarded cells, usually embryonic callus, are then propagated into plants, at least some of which have the bombarded DNA introduced into their genome.

The most important impact of particle bombardment has been to enable transfer of genes into plants normally recalcitrant to other gene transfer techniques. Many of these plants are agronomically important, and include cereals such as rice (Christou et al., 1991, BioTechnology 9 957; International Publication No. W092/20809), wheat (International Publication No. W094/13822) and sugarcane (Franks & Birch, 1991, Aust. J. Plant. Physiol. 18 471).

The earliest version of microprojectile bombardment may be found in European Patent No. 331855, but considerable effort has since been made to refine this powerful and widely-used technique. One such effort has been to improve particle guns, with various incarnations being developed which include helium-driven devices (Sanford et al., 1991, Technique 3 3), a microtargeting particle accelerator (Sautter et al., 1991, BioTechnology 9 1080), a pneumatic apparatus (lida et al., 1990, Theor. AppI. Genet. 80 813) and an electric-discharge type apparatus (Christou et al., 1991, supra).

Other efforts have focussed on broadening the type of cells or tissues which are amenable to microprojectile bombardment, and from which transgenic plants may be propagated. Although embryogenic callus tends to be the tissue type of choice, other tissues such as meristem, zygotic embryos, root and stem sections have also been successfully employed (as reviewed in Christou, Particle Bombardment Technology for Gene Transfer pp 71-99. Eds Ning-Sun Yang & Paul Christou, UWBC Biotechnical Resource Series).

Overall, the aim of genetic engineering of plants has been to transfer two genes: usually a gene of interest together with a selection marker gene. This has been dictated by two considerations

(i) the fact that for the purposes of gene transfer, genes are usually inserted into specialized expression vectors which places practical limitations on the number of genes which can be transferred; and

(ii) the underlying goal of genetic engineering to precisely introduce or modify a specific phenotypic trait by transferring one or a few fully characterized gene(s) which underlie(s) said phenotypic trait.

### OBJECT OF THE INVENTION

It is therefore an object of the invention to provide an improved method of plant gene transfer which utilizes microprojectile bombardment, which method alleviates one or more of the aforementioned deficiencies of the prior art, or at least offers a useful alternative.

### SUMMARY OF THE INVENTION

Therefore, the present invention is broadly directed to gene transfer by microprojectile bombardment where DNA is directly isolated from a donor plant and transferred to cells or tissues of a recipient plant to thereby produce a transgenic plant which, in some cases, may have substantial introgressions of donor genes.

In one aspect, the invention resides in a method of gene transfer which includes the steps of:

(i) transforming a recipient plant cell or tissue by microprojectile bombardment with DNA directly isolated from a donor plant; and

(ii) selectively propagating a transgenic plant from the transformed recipient plant cell or tissue obtained in step (i).

In another aspect, the present invention resides in a recipient plant cell or tissue transformed according to step (i) of the first-mentioned aspect.

Preferably, the recipient plant is a cereal.

More preferably, the recipient plant is a member of the species Oryza sativa and the donor plant is wild rice, such as Zizania palustris.

In yet another aspect, the present invention resides in a transgenic plant produced by the method of the first-mentioned aspect.

In still yet another aspect, there is provided a transgenic plant propagated from microprojectile bombarded cells or tissue, a genome of which transgenic plant has significant introgressions of donor plant genes integrated therein.

Suitably, at least 0.01% of said donor plant genome is integrated therein. Preferably, at least 0.1%, more preferably 1.0% or even more preferably 10% of said donor plant genome is integrated therein.

In a further aspect, the present invention provides flowers, fruit, seeds, grains, pollen, leaves, roots, tubers, cuttings, material suitable for reproductive or vegetative propagation and other cells or tissues of a transgenic plant of the invention.

In a still further aspect, the present invention provides a progeny plant propagated from a transgenic plant of the present invention. In this regard, it will be understood that a transgenic plant of the present invention may be used as a source of genetic material for subsequent breeding or for genetic engineering purposes.

Preferably, the transgenic plant is a cereal.

More preferably, the transgenic plant is a member of the species Oryza sativa.

Preferably, the transgenic plant displays one or more phenotypic traits of the donor plant not normally present in said recipient plant.

Throughout this specification and claims which follow, unless the context requires otherwise, the word “comprise” and variations such as “comprises” or “comprising”, will be understood to imply the inclusion of a stated integer or group of integers or steps but not the exclusion of any other integer or group of integers.

### BRIEF DESCRIPTION OF THE TABLES AND FIGURES

TABLE 1 AFLP Primer Combinations. All primers were provided in the commercially available Perkin-Elmer AFLP Plant Mapping Kit. Primer sequences are proprietary information and could not be obtained for the purposes of this specification. Column headings refer to Mse I primers and row headings refer to Eco RI primers. Numbers refer to primer pairs used as referred to in the specification and Tables.

TABLE 2 Selective amplification conditions for thermal cycling.

TABLE 3 AFLP marker analysis of plant X1-18 using three different primer pairs.

TABLE 4 Analysis of seven individual transgenic plants using AFLP primer pair #1. The total number of AFLP bands present in Z. palustris were 118, of which 60 were Z. palustris-specific.

FIG. 1 Morphological differences in a grain from a transgenic O. sativa plant (centre) compared with a grain from donor Z. palustris (right) and a grain from recipient O. sativa cultivar Jarrah (left).

FIG. 2 AFLP analysis of O. sativa plants genetically-modified according to the method of the invention by bombardment with Z. palustris genomic DNA. a: AFLP profile for O. sativa (Jarrah); b: AFLP profile for transgenic plant X4-8; c: AFLP profile for transgenic plant X4-9; and d: AFLP profile for Z. palustris.

### DETAILED DESCRIPTION OF THE PREFERRED EMBODIMENTS

Prior art gene transfer methods which employ microprojectile bombardment have been exclusively directed to the transfer of one or a few known genes which underlie a known phenotypic trait. The present inventors have realized that this practice has effectively limited the scope of gene transfer as a useful tool in plant breeding, and that it may be desirable to transfer one or more genes from a donor plant having one or more desired phenotypic traits, without actually selectively isolating donor gene(s) responsible for that trait.

This invention is predicated, at least in part, on the discovery that DNA can be isolated directly from a donor plant and transferred to a recipient plant cell or tissue by microprojectile bombardment. Surprisingly, the method of the invention is capable of producing transgenic plants with significant donor plant gene introgressions such as might be achieved by traditional breeding methods, but across taxonomic barriers which are impenetrable to traditional breeding methods.

Suitably, the directly isolated DNA used at step (i) is in the form of genomic DNA or cDNA. It will be appreciated by those skilled in the art that cDNA is complementary DNA synthesized by reverse transcriptase from an isolated RNA template.

General methods applicable to plant genomic DNA and RNA isolation, and PCR inclusive of RT-PCR can be found in Chapters 1 & 3 of of PLANT MOLECULAR BIOLOGY: A Laboratory Manual. Ed. M.S. Clark (Springer-Verlag, 1996) which are herein incorporated by reference.

Preferably, the DNA is genomic DNA.

Advantageously, the DNA is a high-molecular weight fraction of genomic DNA.

A preferred method of genomic DNA isolation may be found in Weining & Langridge, 1991, Theor. Appl. Genet. 82 209, which is herein incorporated by reference.

Throughout this specification and claims “directly isolated” means DNA which has been removed from it's natural state, without the additional step of selecting a particular gene from the isolated DNA for the purpose of gene transfer by microprojectile bombardment. It will therefore be understood that the DNA is in essentially uncharacterized form, but probabilistically, may contain one or a plurality of donor plant genes which underlie one or more desired phenotypic traits.

As used herein, a “gene” is broadly defined as a unit of genetic material which includes and encompasses amino acid coding regions and regulatory elements such as promoters, enhancers, terminators, translation initiation or termination signals and splice acceptor/donor sequences.

The directly isolated may be expected to contain regulatory sequences which direct gene expression, thereby obviating the need for said vector. Thus, although it is preferable that the directly isolated DNA at step (i) is not ligated into a vector, it is nevertheless contemplated that the DNA could be transferred in a form where it is ligated into said vector. In order to insert the directly isolated DNA into said vector, the DNA could be subjected to limited restriction endonuclease digestion and ligated into compatible restriction sites in the vector. Another possibility is that cDNA could be “blunt end cloned” into said vector, or adaptors ligated to the cDNA to assist cloning. These vector cloning steps are standard in the art and exemplary methods are provided in Chapters 2 & 3 of PLANT MOLECULAR BIOLOGY: A Laboratory Manual. Ed. M.S. Clark (Springer-Verlag, 1996) which are herein incorporated by reference.

Suitably, the vector comprises one or more regulatory sequences which direct expression of a DNA sequence operably linked thereto. These regulatory sequences include constitutive and inducible promoters, terminators, polyadenylation signals, translation initiation/termination signals and splice donor/acceptor sites, for example, which initiate, regulate or otherwise direct expression of a DNA sequence to which they are operably linked.

The skilled person will be well aware of a variety of different vectors suitable for this purpose. Such vectors utilize plant-operative regulatory sequences such as the CaMV 35S promoter and terminator (Nagy et al. In: BIOTECHNOLOGY IN PLANT SCIENCE Eds. Zaitlin et al. Academic Press, pp227-235, 1985), the Emu promoter (Last et al., 1991, Theor. Appl. Genet. 81 581), the Ubi promoter (Christensen & Quail, 1996, Transgenic Res. 5 213), the nopaline synthase (nos) promoter/terminator (Harpster et al.,1988, Mol. Gen. Genet. 212 182) and octopine synthase (ocs; MacDonald et al., 1991, Nucl. Acid. Res. 19 5575) terminators, for example. All of the aforementioned are incorporated herein by reference.

Also contemplated are Bacterial Artificial Chromosome (BAC) vectors, such as described in Hamilton, 1997, Gene 200 107, which is herein incorporated by reference.

In light of the foregoing, it will be appreciated that the gene transfer method of the invention is free from the encumbrances created by the prior art practice of transferring fully characterized DNA, with the intention of transferring one or a few characterized gene(s) underlying a known phenotypic trait in order to confer said phenotypic trait upon a transgenic plant. Furthermore, there is no requirement that the DNA be transferred in a vector, such as is typically required, and therefore does not severely restrict the number of genes which can be transferred.

It is preferred that at step (i) a selection construct comprising a selection marker gene operably linked to a plasmid vector is provided together with said directly isolated DNA. Said selection marker may be selected from the group consisting of a hygromycin resistance gene (hmr); a β-glucuronidase (GUS) gene; a neomycin phosphotransferase II (npt II) gene; a phosphinothricin acetyl transferase (bar) gene; a dihydrofolate reductase gene (dhfr) gene; a 5-enolpyruvylshikimate-3-phosphate synthase (epsp) gene; a chloramphenicol acetyl transferase (cat) gene; a 3′-adenylyltransferase (aadA) gene; an acetohydroxyacid synthase (ahas) gene; a nopaline synthase (nos) gene; and a luciferase (lux) gene.

Preferably, the selection marker gene is hmr, which nucleic acid encodes hygromycin phosphotransferase and thereby confers an ability to grow in the presence of normally toxic concentrations of hygromycin.

It will be appreciated that some said selectable marker genes allow selection by addition of selection agents such as hygromycin, kanamycin or G418 (npt II) or glyphosate (epsp), while others allow selection via screening of tissue samples, such as lux and GUS. A more detailed discussion of selectable marker genes and appropriate selective agents is provided in Christou, Particle Bombardment Technology for Gene Transfer pp 71-99. Eds Ning-Sun Yang & Paul Christou, UWBC Biotechnical Resource Series.

Suitably, the selectable marker gene is ligated into a plasmid vector to form a selection construct. Typically, the plasmid vector contains one or more regulatory sequences as hereinbefore defined.

A preferred plasmid vector for this purpose is pGL2.

In principle, the method of the invention is applicable to transfer of DNA between any donor and recipient plant, although it will be understood by the skilled person that successful transfer and stable integration of DNA generally becomes more difficult the further the donor and recipient plant are separated taxonomically.

In one embodiment, said donor plant and said recipient plant are members of distinct species.

In another embodiment, said donor plant and said recipient plant are members of distinct genera.

In one particular embodiment, said donor plant and said recipient plant are cereals.

In another particular embodiment, said donor plant is Zizania palustris (wild rice) and said recipient plant is Oryza sativa (rice).

Suitably, said recipient plant cell or tissue is amenable to microprojectile bombardment, and is capable of being selectively propagated into a transgenic plant. Preferably, said cell or tissue is embryogenic callus, although a variety of other cells or tissues may be applicable, including meristem, zygotic embryos, root and stem sections. A detailed discussion of this topic may be found in Christou, Particle Bombardment Technology for Gene Transfer pp 71-99. Eds Ning-Sun Yang & Paul Christou, UWBC Biotechnical Resource Series for example.

Suitably, said microprojectiles are in the form of small, biologically-inert particles such as gold or tungsten (M17 grade, as for example used in Wan & Lemaux, 1994, Plant Physiol. 104 37, which is herein incorporated by reference) as are well known in the art. Preferably, said microprojectiles are in the form of 1 μM gold particles. Methods of immobilizing the directly isolated DNA to microprojectiles are well known in the art, and a detailed description of a preferred method based on that of Sanford et al., 1993, Meth. Enzymol. 217 483 (which is herein incorporated by reference), is provided hereinafter.

As will be described in detail hereinafter, the transgenic plants of the present invention may have as few one donor gene integrated into their genome, or may have significant introgressions of donor genes. This may be the case even where the donor and recipient plants share only limited genetic similarity and have proven resistant to traditional breeding methods. The present invention therefore provides a method of producing transgenic plants unattainable by conventional breeding or genetic engineering techniques.

In an embodiment, a genome of said transgenic plant has at least 0.01%, preferably at least 0.1%, more preferably at least 1.0%, or even more preferably at least 10.0% of said donor plant genome integrated therein.

In order to estimate the proportion of said donor genome integrated into the genetically-modified plane genome, it is preferred that Amplified Fragment Length Polymorphism (AFLP) analysis is used (Vos et al., 1995, Nucl. Acid. Res. 23 4407, which is herein incorporated by reference).

However, other methods are also contemplated. These include Rapid Amplification of Polymorphic DNA (RAPD; Williams et al., 1990, Nucl. Acid. Res. 18 6531, which is herein incorporated by reference), Restriction Fragment Length Polymorphism (RFLP; Nathans & Smith, 1975, Ann. Rev. Biochem. 46 273, which is herein incorporated by reference) and microsatellite analysis, such as described in Roder et al., 1995, Mol. Gen. Genet. 246 327 and reviewed in Powell et al., 1996, Trends Plant Sci. 7 215, which are both herein incorporated by reference.

An example of an estimate by AFLP analysis is provided hereinafter. A generalized hypothetical example follows the reasoning that if a donor plant genome comprises × genetic markers unique thereto, and a transgenic plant selectively propagated from bombarded callus comprises 0.03 × said genetic markers, then the proportion of donor plant genome integrated therein equates to 3%. Such an estimate assumes that on average, each said genetic marker is linked to an equal sub-portion of the donor genome, that each said genetic marker is equally capable of integration into the recipient genome, and that each said genetic marker is equally detectable once so integrated. In the absence of evidence to the contrary, these assumptions are maintained for the purposes of this specification.

In cases where the skilled person wishes to identify a specific gene or genes of interest integrated into the genome of a transgenic plant of the invention, a variety of well-known techniques are suitable for this purpose. These include PCR analysis and hybridization techniques such as Southern blotting and Northern blotting, or protein-based methods such as immunoblotting and immunoprecipitation. In this regard, the skilled person is referred to Chapters 1, 3, and 9 of PLANT MOLECULAR BIOLOGY: A Laboratory Manual. Ed. M.S. Clark (Springer-Verlag, 1996) which are herein incorporated by reference.

So that the invention can be understood in more detail reference is made to the following non-limiting experimental examples.

### EXPERIMENTAL

1. MATERIALS AND METHODS

1.1 Preparation of genomic DNA

Zizania palustris (wild rice) leaf material was obtained from commercial field crops in southern New South Wales, Australia. DNA was isolated from approximately 5 gram of young leaves essentially according to the procedure of Weining & Langridge, 1991, supra.

1.2 Transformation

Embryonic callus cultures from the rice cultivar Jarrah were derived from mature seeds and used as targets for microprojectile bombardment by a particle inflow gun (Finer et al., 1992, Plant Cell Rep. 11 323). The generation of embryonic callus suitable for bombardment, and the bombardment procedure using the DuPont Biolistics PDS-1000/He apparatus, are described in detail in Abedenia et al., 1997, Aust. J. Plant Physiol. 24 133, which is herein incorporated by reference. Briefly, 8 μg of high molecular weight Z. palustris genomic DNA and 2 μg of plasmid pGL2 (Shimamoto et al., 1989, Nature 338 274) encoding the hygromycin-resistance (hmr) gene (at a 1:1 molar ratio) in 10 μl was immobilized on microprojectiles in the form of 1 μM gold particles, by a method originally described by Sanford et al., 1993, supra. 5 μl of this microprojectile preparation was used per shot by the particle inflow gun. Optimal conditions for microprojectile bombardment were established using the GUS (β-glucuronidase) system as previously described (Abedenia et al., 1997, supra).

To enhance embryogenesis, callus was cultured on osmotic medium (MSC together with 0.2 M sorbitol and 0.2 M mannitol; Vain et al., 1993, Plant Cell Reports 12 84) for 4 hrs prior to bombardment, and following bombardment, was transferred to MSC medium supplemented with 30-50 mg.L⁻¹ Hygromycin B (Sigma). Selection was applied throughout callus proliferation, regeneration and plant propagation. Once selectively propagated plants had reached 10-15 cm in height, they were transferred to soil in a glasshouse.

1.3 Analysis of gene transfer

In order to semi-quantitatively monitor the extent of genome transfer into selectively-propagated plants, Amplified Fragment Length Polymorphism (AFLP) analysis was performed (Vos et al., 1995, supra). AFLP analysis was conducted on genomic DNA extracted from 200 mg of leaves (extract made as previously described) obtained from plants selectively propagated from microprojectile-bombarded callus. Reactions were performed using a Perkin Elmer AFLP Plant Mapping kit according to the manufacturers instructions (PE Applied Biosystems: AFLP Plant Mapping, 1996, pp 2-31, which is herein incorporated by reference). A brief synopsis of the AFLP analysis method is provided in sections 1.3.1 to 1.3.5.

1.3.1 Preparation of penomic DNA

Extracted DNA was digested with the restriction endonucleases Mse I and Eco RI, and Mse I and Eco RI adaptors ligated in a single reaction for 2 hr at 37° C. Adaptor-ligated DNA was then diluted in TE and stored at −20° C.

1.3.2 Adaptors and Primers

All adaptors and primers were supplied by the manufacture, and only limited primer and adaptor sequence information is provided by the manufacturer. Table 1 lists all of the primer combinations provided by the manufacturer and those used in this study.

1.3.3 Preselective Amplification

Preselective amplification reduces the overall complexity of amplification by making target sequences the predominant species. The sequence of the adaptors, Mse I and Eco RI restriction sites and sequences adjacent to the restriction sites serve as primer binding sites for preselective amplification. Reactions involved use of AFLP Preselective Primer pairs provided by the manufacturer to amplify from the adaptor-ligated DNA prepared at 1.3.1. Thermal cycling was performed using PE 9600 Thermal Cycler under the following conditions

1 cycle at 720° C. for 2 min; then 20 cycles at 94° C. for 1 min; 56° C. for 0.5 min and 72° C. for 2 min; then

1 cycle at 60° C for 30 min; then hold at 4° C. Preselective amplification products were diluted in TE and stored at 4° C.

1.3.4 Selective Amplification

Reactions were performed using Preselective amplification products, Mse I primer (5 μM) and Eco RI dye (FAM) primer (1 μM) together with manufacturer-supplied reaction mix. Thermal cycling was performed using a PE 9600 Thermal Cycler according to the cycling conditions shown in Table 2.

1.3.5 Detection of AFLP products

Detection of AFLP fragments containing the FAM-labeled Eco RI primer was achieved by capillary electrophoresis using an ABI Prism 310 GeneAnalyser. The data were analysed using dedicated GeneScan Analysis and Genotyper software.

2. RESULTS & DISCUSSION

Introgression of genes from plant-to-plant is usually limited to plants of the same species or genus (Harlan & de Wet, 1971, Taxon 20 509). Although in rice, interspecific hybrids between Qryza species may produce fertile offspring (Naredo et al., 1997, Genet. Res. Crop Evol. 44 17), hopes that more distant taxa may have genetic material which might be introduced into Oryza and confer improvements which are both stable, and heritable have not been realized. In this regard, reported hybrids between rice and the distant relative Portersia coarcata, recovered by embryo rescue, were not fertile (Jenna, 1994, Curr. Sci. 67 744).

Rice was the first cereal to successfully be transformed, in which case transformed rice plants were regenerated from transformed protoplasts (Shimamoto et al., 1989, Mol. Breed. 4 99). Subsequent protocols have been developed which enable rice transformation by microprojectile bombardment of callus derived from mature embryos of commercial rice cultivars (Christou, 1997, Plant. Mol. Biol. 35 197; WO 92/20809; Abedenia et al., 1997, supra). Traditional transformation by microprojectile bombardment has focussed on the transfer of a single gene of interest, usually in combination with a selectable marker gene. For example, the method disclosed in International Publication No. WO92/20809 described transformation with the Bar gene inserted into the plasmid pCMC2114 which encodes the GUS marker.

Although, the present inventors used microprojectile bombardment as a starting point for genome transfer into rice, their aim was not to merely transfer a fully-characterized single gene, but to transfer directly-isolated donor DNA which represents a substantial portion of a donor plant genome. The intended purpose was to genetically-modify plants so as to display one or more genetically-encoded characteristics of the donor. Essentially, this could amount to creation of a genetically-modified “hybrid”, by an approach entirely distinct from the traditional process of cross-breeding and outside the capabilities of conventional microprojectile bombardment approaches. Importantly, although the donor plant Z. palustris was known to display distinct phenotypic traits such as cold-tolerance, the genetic basis for this remains a mystery.

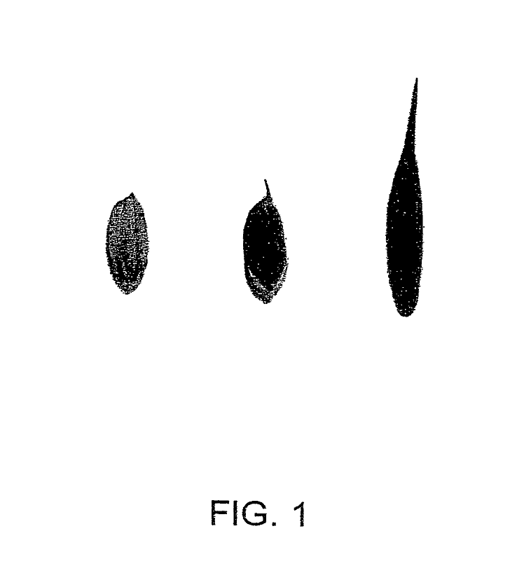

The present inventors isolated high-molecular weight genomic DNA from the wild-rice species Z. palustris (donor) as a representative sample of the genome of Z. palustris for transfer into O. sativa (recipient) callus. The genomic DNA was not inserted into a plasmid vector, but rather was directly immobilized (together with a plasmid encoding hygromycin phosphotransferase) onto 1 μM gold particles. Transgenic plants were regenerated from bombarded callus under conditions which selected for resistance to hygromycin. Fifteen (15) bombardments of 20 callus targets resulted in the successful regeneration and transfer to the glasshouse of more than 250 rice plants. The plants showed considerable morphological variation consistent with the introgression of genes from Z. palustris. An example of morphological variation between recipient (Jarrah) and transgenic plants is shown in FIG. 1.

The transgenic plants varied greatly in height, which characteristic is not normally observed in transgenic plants. Variations in grain colour and shape were observed (FIG. 1), with the darker colour characteristic of Z. palustris being evident to varying degrees in the grains of transgenic progeny. Several transgenic lines had grains intermediate in length between the shorter O. sativa grains and the longer Z. palustris grains. Also noted was the fact that several transgenic lines had long awns characteristic of Z. palustris.

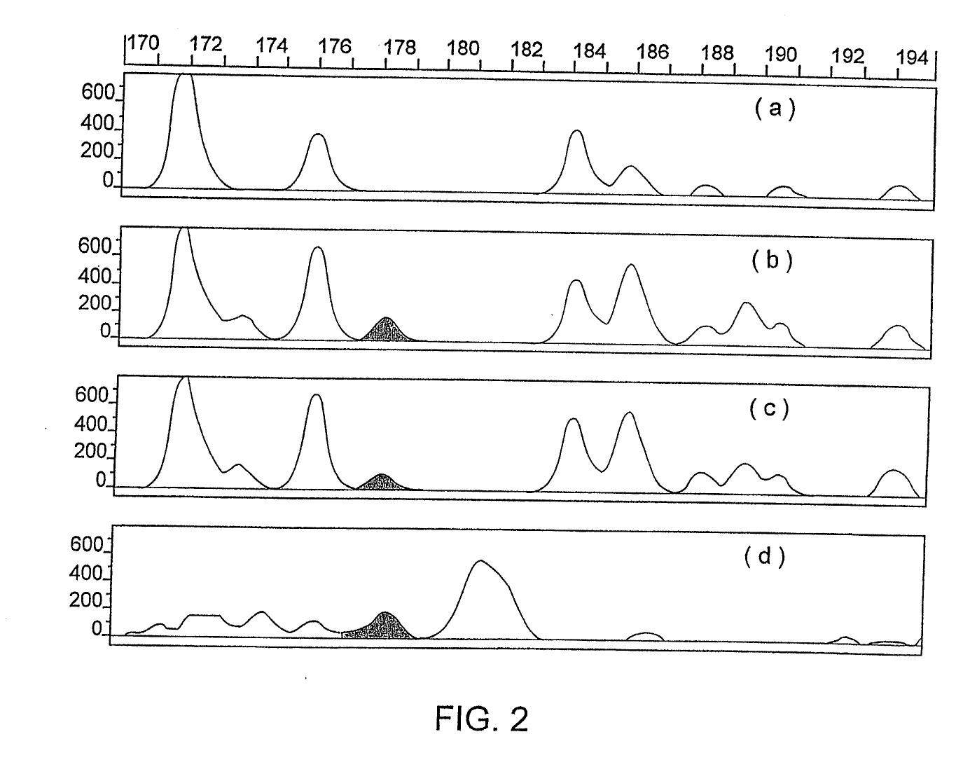

A typical AFLP analysis using capillary electrophoresis and fluorescence detectionis shown in FIG. 2. AFLP analysis indicated the presence of Z. palustris DNA in a significant proportion of plants tested. Analysis of 151 plants with AFLP primer pair #1 demonstrated the presence of 10 different AFLP bands from Z. palustris in 7 different individuals (see Table 4). The range of introgression was from 1.7% to 6.7%. That is, 1.7% to 6.7% of Z. palustris-specific AFLP markers were present in the 7 individuals tested, suggesting that in a number of plants genetically-modified according to the method of the invention, a significant portion of the donor plant genome had been transferred.

Further analysis of 37 individual plants, chosen on the basis of their unusual appearance, with AFLP primer pair #4 revealed 20 Z. palustris-derived AFLP bands in 9 of the 37 plants. As shown in Table 3, one O. sativa plant had 16 of a possible 122 Z. palustris-specific AFLP markers identifiable following amplification with three different primer pairs. This equates to approximately 13% of the Z. palustris genome being transferred to this plant and strengthens the conclusion that many of the transgenic plants of the invention may contain significant introgressions of the Z. palustris genome.

However, it should be noted that control experiments indicated that a small number of new markers were being generated due to genetic change during the transformation process. Analysis of 4 control plants with primer pair #4 revealed a single novel AFLP product which had no apparent counterpart in Z. palustris. Fertility of the transgenic plants varied, but all plants set some seed. The level of introgression suggests that screening of moderate sized populations might allow the recovery of rice plants with preferred phenotypic traits. In principle, populations of hundreds or thousands of plants can be generated by the method of the invention and used as a modified germplasm resource. Detailed molecular analysis of each transgenic rice plant will be necessary to establish the number of sites of insertion of Z. palustris DNA and the size of Z. palustris DNA inserts. Southern blotting was not a reliable method for analysis of introgression because of the relatively high sequence similarity between Z. palustris and O. sativa DNA interfering with hybridization.

Wild members of Oryzae have been shown to be important sources of genes for improvement of yield (Xiao et al., 1996, Nature 384 223). However, introgression of genes from wild relatives such as Z. palustris to O. sativa has not been possible due to the considerable phylogenetic distance between such family members. In fact, this “distance limitation” constitutes a fundamental barrier to creating hybrid plants throughout the plant world (Sharma, 1995, Euphytica 82 43). Given the success of the present invention with regard to Z. palustris, other relatives of rice at a similar phylogenetic distance from O. sativa may be applicable to the method. Such a relative might be Potamophila parviflora.

Gene transfer by microprojectile bombardment has generally shown only minimal alteration of the rice genome, suggesting that the introduction of new genes can be achieved while maintaining genetic integrity (Arencibia et al., 1998, Mol. Breed. 4 99). Thus, in commercial crops such as O. sativa which can display low levels of genetic diversity, major genetic introgression from wild relatives should not destroy desirable existing traits, but should improve genetic diversity and enhance resistance to pests and other environmental contingencies.

Gene transfer by microprojectile bombardment has traditionally involved the transfer of one or two genes (usually a gene of interest together with a selectable marker) into cells or tissues derived from a recipient plant. This technique has therefore been used to transfer a known gene of interest for the purpose of transferring a particular trait. The present invention expands the usefulness of microprojectile bombardment as a means of gene transfer into the realm of creating hybrid plants. In principle, the donor plant genome may essentially be uncharacterized, but because of the significant introgression of donor plant genes according to the method of the invention, there is high probability that a number of genes are transferred to the genetically modified plant, which genes may underlie desirable phenotypic traits.

TABLE 1

| | | | | | | | |
|---|---|---|---|---|---|---|---|
| —CAA | —CAC | —CAG | —CAT | —CTA | —CTC | —CTG | —CTT |

| | | | | |
|---|---|---|---|---|
| —AA | | | #3 | |
| —AC | | | | |
| —AG | | | | |
| —TA | | | | #2 |
| —TC | | | | |
| —TG | #4 | #1 | | |
| —TT | | | | |

TABLE 2

| | | | NO. OF |
|---|---|---|---|
| HOLD | CYCLE | HOLD | CYCLES |
| 94° C. for 2 min | 65° C. for 30 sec | 72° C. for 2 min | 1 |
| 94° C. for 1 sec | 64° C. for 30 sec | 72° C. for 2 min | 1 |
| 94° C. for 1 sec | 63° C. for 30 sec | 72° C. for 2 min | 1 |
| 94° C. for 1 sec | 62° C. for 30 sec | 72° C. for 2 min | 1 |
| 94° C. for 1 sec | 61° C. for 30 sec | 72° C. for 2 min | 1 |
| 94° C. for 1 sec | 60° C. for 30 sec | 72° C. for 2 min | 1 |
| 94° C. for 1 sec | 59° C. for 30 sec | 72° C. for 2 min | 1 |
| 94° C. for 1 sec | 58° C. for 30 sec | 72° C. for 2 min | 1 |
| 94° C. for 1 sec | 57° C. for 30 sec | 72° C. for 2 min | 1 |
| 94° C. for 1 sec | 56° C. for 30 sec | 72° C. for 2 min | 23 |
| 60° C. for 30 min | — | — | 1 |
| 4° C. hold | — | — | — |

TABLE 3

| | | TOTAL Z. | |
|---|---|---|---|
| | NUMBER OF | palustris | |
| | AFLP | SPECIFIC | % OF Z. |
| AFLP PRIMER | MARKERS | AFLP | palustris AFLP |
| PAIR | IDENTIFIED | MARKERS | MARKERS |
| #1 | 3: (171 bp; 211 | 60 | 5 |
| | bp; 220 bp) | | |
| #2 | 4: (82 bp; 111 | 33 | 12 |
| | bp; 148 bp; 496 | | |
| | bp) | | |
| #3 | 9: (221 bp; 307 | 29 | 31 |
| | bp; 329 bp; 339 | | |
| | bp; 348; 356 | | |
| | bp; 384 bp; 396 | | |
| | bp; 466 bp) | | |
| TOTAL: | 16 | 122 | 13 |

TABLE 4

| | | % OF Z. |
|---|---|---|
| | | palustris |
| | NUMBER OF AFLP MARKERS | AFLP |
| PLANT NO. | IDENTIFIED | MARKERS |
| X1-18 | 3; 171 bp; 211 bp; 220 bp | 5.0 |
| X1-37 | 1; 212 bp | 1.7 |
| X4-8 | 1; 372 bp | 1.7 |
| X4-9 | 1; 372 | 1.7 |
| X5-31 | 3; 171 bp; 242 bp; 248 bp | 5.0 |
| X5-39 | 2; 171 bp; 248 bp | 3.3 |
| X5-48 | 4; 171 bp; 248 bp; 416 bp; 442 bp | 6.7 |

1. A method of gene transfer including the steps of:
(i) transforming a recipient plant cell or tissue by microprojectile bombardment with DNA directly isolated from a donor plant; and
(ii) selectively propagating a transgenic plant from the transformed recipient plant cell or tissue obtained in step (i).

2. The method of claim 1, wherein a selection construct is included at step (i).

3. The method of claim 1 wherein the DNA is genomic DNA.

4. The method of claim 3 wherein the DNA is a high molecular weight fraction of genomic DNA.

5. The method of claim 1 wherein the donor plant and the recipient plant are members of different genera.

6. The method of claim 1 wherein the donor plant and the recipient plant are members of different species.

7. The method of claim 1 wherein the donor plant and the recipient plant is a cereal.

8. The method of claim 7 wherein the donor plant is of the species Zizania palustris.

9. The method of any preceding claim wherein the recipient plant is of the species Oryza sativa.

10. A recipient plant cell or tissue transformed according to step (i) of claim 1.

11. A transgenic plant, a genome of which has at least 0.01% of a genome of a donor plant integrated therein.

12. The transgenic plant of claim 11, wherein said genome has at least 0.1% of said donor plant genome integrated therein.

13. The transgenic plant of claim 12, wherein said genome has at least 1.0% of said donor plant genome integrated therein.

14. The transgenic plant of claim 13, wherein said genome has at least 10% of said donor plant genome integrated therein.

15. A transgenic cereal plant according to any one of claims 10-14.

16. A transgenic O. sativa plant according to any one of claims 10-14.

17. A transgenic plant produced according to the method of any one of claims 1-9.

18. A transgenic plant according to any one of claims 11-17, which transgenic plant has one or more phenotypic traits of said donor plant not normally present in said recipient plant.

19. A fruit or grain obtained from the transgenic plant of any one of claims 11-18.

20. A seed obtained from the transgenic plant o f any one of claims 11-18.

21. A progeny plant propagated from the transgenic plant of any one of claims 11-18.
